(19) United States
(12) Patent Application Publication
(10) Pub. No.: US 2011/0232134 A1
(43) Pub. Date: Sep. 29, 2011

(54) ASYNCHRONOUSLY VIBRATING DEVICE FOR USE WITH FOOTWEAR AND METHODS OF USE

(75) Inventors: Christopher L. Radl, Malvern, PA (US); Robert Kropp, Norristown, PA (US)

(73) Assignee: BOEHRINGER LABORATORIES LLC, Phoenixville, PA (US)

(21) Appl. No.: 13/050,511

(22) Filed: Mar. 17, 2011

Related U.S. Application Data
(60) Provisional application No. 61/316,952, filed on Mar. 24, 2010.
(60) Provisional application No. 61/374,821, filed on Aug. 18, 2010.

(51) Int. Cl. A61F 5/14; A61H 1/00

(52) U.S. Cl. 36/141; 601/46

(57) ABSTRACT

A device and methods of use for applying vibration, e.g., asynchronous vibration, to the foot of a person is disclosed. The device includes an insole for disposition within an item of footwear and an associated asynchronous vibration inducing mechanism. The insole is a thin, conformable, base member having a pair of wings, each of which includes at least one motor (e.g., brushless electrical motor) forming a portion of the vibration inducing mechanism. The base member is formed of a material, e.g., a resin bonded non-woven layer, which exhibits good vibration transmission characteristics. A power source (e.g., at least one battery) is coupled to the motors to cause them to vibrate when operated, whereupon asynchronous vibrations are produced by the motors, which are transmitted and propagated via the wings to the base member to apply asynchronous vibrations across the sole of the wearer's foot.

### CROSS-REFERENCE TO RELATED APPLICATIONS

This application claims priority from Provisional Application Ser. No. 61/316,952, filed on Mar. 24, 2010, entitled Vibrating Insole Device And Method Of Use, and Ser. No. 61/374,821, filed on Aug. 18, 2010, entitled Asynchronously Vibrating Device For Use With Footwear And Methods Of Use, which applications are assigned to the same assignee as this application and whose disclosures are incorporated by reference herein.

### STATEMENT REGARDING FEDERALLY SPONSORED RESEARCH OR DEVELOPMENT

“Not Applicable”

### INCORPORATION-BY-REFERENCE OF MATERIAL SUBMITTED ON A COMPACT DISK

“Not Applicable”

### FIELD OF THE INVENTION

This invention relates generally to medical devices and more particularly to devices for use in footwear to provide asynchronous vibratory motion to the wearer's foot for relief of pain.

### BACKGROUND OF THE INVENTION

According to the National Pain Foundation, chronic pain lasting more than three months affects as many as 70 million Americans and is noted as the most costly health problem in the United States. Additionally, one in four Americans suffers from pain episodes that last longer than 24 hours. While most forms of acute pain tend to resolve over time, chronic pain may never resolve and can be debilitating for some patients. Chronic pain can be associated with many conditions, including but not limited to, trauma, diabetes, autoimmune disorders, tumors, heredity and toxins.

Often there is no exact cure for chronic pain; however, the symptoms can be managed to some extent. If there is an underlying disease, treatment of this disease can often result in a reduction of symptoms. Unfortunately, as time progresses, medications have been known to lose their effectiveness and symptoms worsen. Additionally, some forms of pain are not alleviated with any medications. In fact, typical medications prescribed for the treatment of chronic pain only have 30%-50% effectiveness. Additionally, these medications have been associated with such severe side effects that patients refuse to take them or only take them when the pain is unbearable.

Pain experienced by individuals varies, not only from person to person but also in intensity and location. When pain inflicts the feet, the discomfort may be so bad that a simple task such as walking or even sitting still can become an impossible task. Pain signals originate in the peripheral nerves of the feet with nociceptors. These signals are then transferred to the spinal cord and ultimately to the brain. Throughout this pathway there are specialized nerve cells that act as “gates” in order to control and filter the signals that go to the brain.

Pain signals are initiated at the site of injury, or in the case of most individuals suffering from peripheral neuropathic pain, in their feet. These signals originate at mechanoreceptors located in the various layers of the skin and muscle. The pain signals are transmitted along C-afferent fibers to the dorsal horn of the spinal column. These pathways in the dorsal horn act as the primary gates for the transmission of pain to the brain. The C-afferent fibers are not myelinated and therefore have a slow conduction speed; less than 2 m/s.

With some patients, pain can be managed to an extent. For severe pain, opioid pain relievers are a common form of treatment. These medications effectively block pain by causing a release of enkephalin and dynorphin-containing neurones in the dorsal horn. These molecules bind to opioid receptors located on the C-fiber terminals in the dorsal horn of the vertebrae and effectively block pain from passing through the gate.

Many individuals suffer from chronic foot pain as a result of nerve damage. Because the nerves are damaged they may send pain signals to the brain. This type of nerve damage is referred to as peripheral neuropathy and can be significant and often debilitating. With peripheral neuropathy affecting the feet, the nerves have been damaged generally as a result of another underlying condition. For example, there is a high incidence of peripheral neuropathy among individuals with diabetes and also occurs as a side effect of chemotherapy in the treatment of cancer. Peripheral neuropathy manifests itself in a number of ways. Individuals may experience a loss of sensation or numbness in their feet. They may also experience chronic pain that manifests itself as a shooting pain or burning sensation. Many individuals experience both conditions. The loss of sensation by persons having peripheral neuropathy can have an adverse impact on balance and can result in falls, etc. Loss of sensation can also make it hard to carry out normal activities such as driving a car, etc.

For minor aches and pains, a common reaction is to rub or massage the injured area. This massaging action, initiates a similar pain blocking cascade in the dorsal horn of the spinal cord, except on a much smaller scale.

Similarly, the application of vibration to primary afferent nerve fibers at the location of pain can also trigger inhibitory signaling pathways in the dorsal horn of the vertebrae. Vibratory stimulation activates mechanoreceptors that send signals via A-beta and A-delta afferent fibers. The signals in these afferent fibers travel around 70 m/s and 11 m/s respectively. Their fast conduction velocity is due to the fact that the overall diameters of the fibers are larger than the diameter of the C-fibers, their axons are myelinated and they have a low activation threshold. In essence, signals initiated by the application of vibration to the foot travel faster to the spinal cord and block pain from being processed.

The mechanoreceptors that activate A-afferent fibers respond to various vibratory frequencies, ranging anywhere from 20 Hz to over 300 Hz. The various frequencies allow individuals to discern between touch, pressure, vibration, proprioception and other forms of sensation. In order to stimulate many mechanoreceptors, it is important to vary the frequency that is applied to the afferent fibers. Applying stimulation at only one or very close frequencies can decrease the efficiency of stimulating neurons over time. Individuals who were exposed multiple times to cutaneous vibration lasting only a few minutes experienced a decrease of perceived intensity in the applicable region.

Additionally, the beneficial effects of combining vibration therapy with movement have been studied. In research on the mechanisms of pain relief by vibration and movement, subjects reported that vibration and movement combined helped to increase their pain threshold, reduce the amount of pain felt and prolong the reaction time of the subject to a painful stimulus. Combining the two treatment modalities further enhanced the pain inhibitory cascade in the dorsal horn of the vertebrae, thereby reducing perceived pain.

Medical devices to treat neuropathic pain are available. One such device is a transcutaneous electrical nerve stimulation (TENS) device. With TENS an electrical current is sent through a part of the body via electrodes. It is believed that the applied electrical current stimulates the nerves in a manner that blocks the pain signals to the brain, thus providing temporary relief from the pain. TENS works for some individuals however it does not work for others. Many find that it produces a somewhat unpleasant sensation. Moreover, TENS is also not that convenient to use. It involves applying electrodes directly to the skin. Often a gel or cream is needed for electrical conductivity. For foot pain, an electrode may need to be placed on the bottom of the foot. If a person needs to walk the electrode has to be removed so that it is not walked upon.

Research in pain reduction through the application of vibration has been studied extensively. Vibration is often compared to the application of transcutaneous electrical nerve stimulation (TENS) in its effectiveness; however, in some cases vibration has been shown to be even more effective. Successful treatment with vibration was found to possess the following characteristics:

1) The applied frequency of vibration is the most effective between 50 and 250 Hz.

2) Mechanical vibration works best when coupled directly to the location of the pain.

3) Therapy with vibration should last between 25 and 45 minutes per session. Residual analgesic effects were noted in many patients after following these recommendations. In some patients, pain relief lasted for almost 18 hours after the application of vibration.

Historically vibration in the form of massage has been used to help with muscle aches and pains. Devices for accomplishing such massages are usually hand held devices that can be placed against sore spots on the body. For foot pain there are massaging devices that one can rest their feet on. Vibrating shoes and insoles have been made to massage the feet. Insoles that create an undetectable vibration to improve balance have also been made.

The use of such devices appears unsuitable for the treatment of neuropathic pain. In particular, stand alone devices are not convenient to use as they generally involve removing ones shoes and remaining seated or stationary during the duration of use. Vibrating shoes are impractical because there are thousands of types and styles of shoes that various individuals chose to wear and incorporating vibration in to all of them is not feasible. In theory vibrating insoles might present the best option, however, devices thus far suffer from the fact that insoles need to be thin and comfortable in order to be used for walking, etc. and to fit in shoes or other footwear. Additionally, the repetitive load placed on insoles due to walking is considerable.

Insoles that incorporate vibration devices all have the vibration generators positioned so that they are beneath the soles of the feet, so in essence a person is walking on the vibration mechanisms. The fact that insoles must be thin significantly limits any type of vibration mechanism that can be placed below a person's foot. If a substantial vibration mechanism is used in an insole it could create significant areas of increased pressure on the foot that could result in injury. Alternately, if the insole was to include adequate soft material to protect the foot from pressure due to standing on a vibration mechanism, it would be too thick to be a practical insert that could be fit into various shoes or other footwear.

Another drawback of prior art vibratory insoles is that the vibrations that they provide are generally of a fixed frequency and amplitude so that the wearer adapts to the vibration, thereby lessening, if not eliminating its beneficial effect.

Thus there presently exists a need for an insole, or other device (e.g., a stocking), that can be placed on the sole of a foot in a shoe and which provides effective therapeutic vibration to the foot.

The subject invention addresses that need.

### SUMMARY OF THE INVENTION

In accordance with one aspect of the invention there is provided a device for applying vibration to the foot of a person. The device is in the form of a member arranged for disposition within an item of footwear worn by the person and basically comprises a thin base member (e.g., an insole) and an asynchronous vibration inducing mechanism (e.g., plural electrically operated motors). The thin body member is formed of a material that exhibits good vibration transmission characteristics and has a medial side, a lateral side and a first wing portion extending from one of the medial side or the lateral side. The base member is arranged to be disposed under the sole of the person's foot, with the first wing portion being located beside a respective portion of the person's foot, but not under the sole of the person's foot. The asynchronous vibratory inducing mechanism is coupled to the first wing portion to cause the first wing portion to vibrate asynchronously, whereupon the asynchronous vibration of the first wing portion is transmitted to the thin base member and propagated thereby to apply asynchronous vibration across the sole of the person's foot.

In accordance with another aspect of this invention there is provided a method for providing vibration to the foot of a person, e.g., to treat peripheral neuropathy, while the person is ambulatory wearing an item of footwear. The method basically entails providing a device, e.g., insole, like that described above and disposing it under the sole of the person's foot, so that the first wing portion is located beside a respective portion of the wearer's foot, but not under the sole of the person's foot, and operating the vibration inducing mechanism to cause the first wing portion to vibrate, whereupon the vibration of the first wing portion is transmitted to the thin body member to apply vibration across the sole of the person's foot.

In accordance with still another aspect of this invention there is provided a method for improving the sense of feel and balance of a person having peripheral neuropathy by providing vibration to the person's foot at a level which is perceptible by the person. One exemplary method of that aspect of the invention entails providing a device comprising a thin base member and an asynchronous vibration inducing mechanism and disposing the base member under the sole of the person's foot. The device is then operated so that the asynchronous vibration inducing mechanism transmits asynchronous vibrations to the base member which propagates the asynchronous vibration thereacross to thereby apply asynchronous vibration across the sole of the wearer's foot.

### DESCRIPTION OF THE DRAWING

FIG. 1 is a top plan view of one exemplary embodiment of a vibrating insole forming a portion of a device constructed in accordance with this invention for providing asynchronous vibration to the foot of a person;

FIG. 2 is an enlarged cross sectional view taken along line 2-2 of FIG. 1;

FIG. 3 is a reduced isometric view of an exemplary power source forming another portion of the device of this invention for effecting the vibration of the insole shown in FIG. 1;

FIG. 4 is an isometric view of the vibrating insole in the position in which it would be installed in a shoe or other item of footwear;

FIG. 5 is an isometric view showing one complete exemplary device constructed in accordance with this invention, i.e., the power source and the insole with the cables connecting them;

FIG. 6 is an isometric view, similar to FIG. 5, but showing the device with the power source mounted adjacent the ankle of a wearer and the insole within the shoe of the wearer;

FIG. 7 is a chart comparing the transmission of the vibratory effect produced by an insole constructed in accordance with this invention to a conventional gel insole and a conventional foam insole; and

FIG. 8 is a graph of a waveform representing the resulting asynchronous vibration produced by an exemplary device constructed in accordance with this invention.

### DETAILED DESCRIPTION OF THE PREFERRED EMBODIMENT

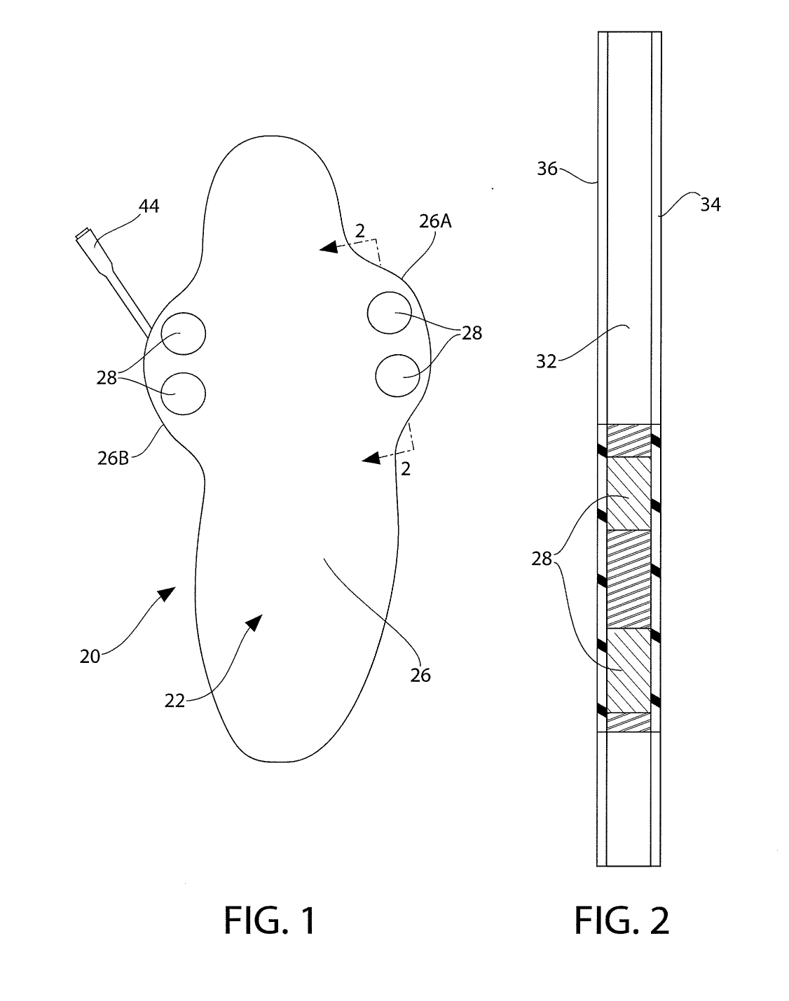

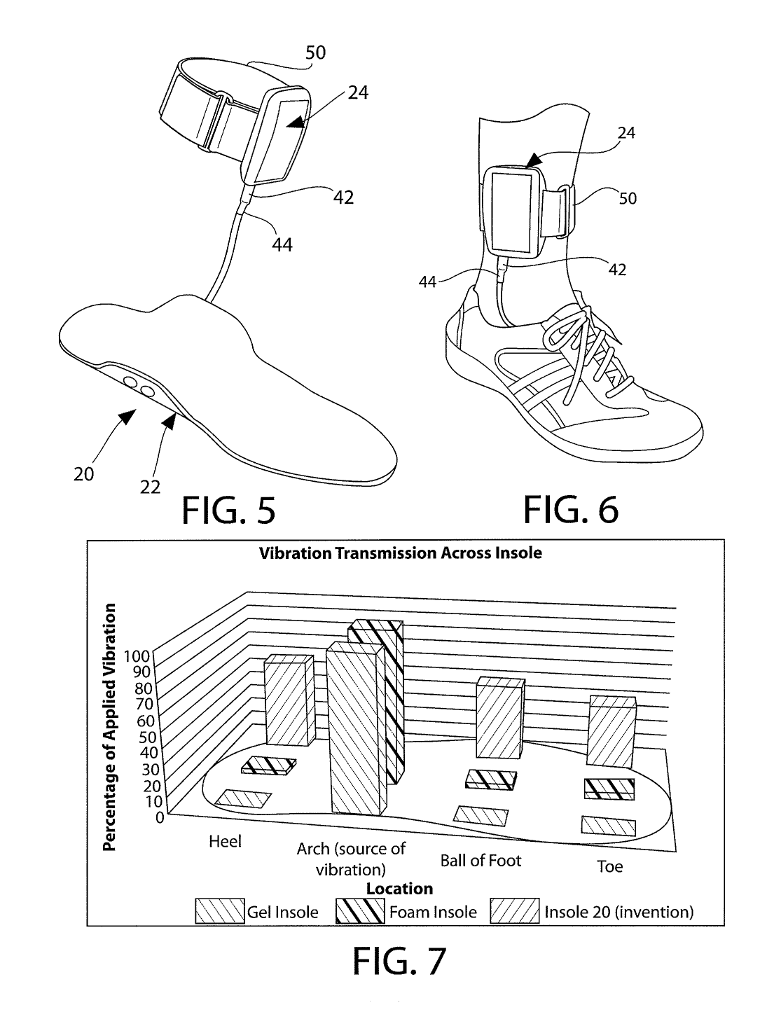

Referring now to the various figures of the drawing wherein like reference characters refer to like parts, there is shown at 20 in FIGS. 1, 5 and 6 at 20 a device for providing asynchronous therapeutic vibration to the foot for pain relief. The device 20 will be described in detail later. Suffice it for now to state that it basically comprises any thin member 22 that is arranged to be disposed in an article of footwear under the sole of the wearer's foot and a power source 24 for causing the thin member to vibrate. In the exemplary embodiments shown the device comprises an insole 22, but may be in some other form, e.g., a sock or stocking. In fact, the device may be incorporated into the article of footwear itself. In all cases the thin member, e.g., insole, 22 of the device 20 includes an asynchronous vibration inducing mechanism (to be described in detail later) which when energized by the power source produces asynchronous vibrations from locations laterally of the sole of the wearer's foot, but which are propagated across the sole of the user's foot.

In the exemplary embodiment shown the asynchronous vibration inducing mechanism comprises plural, e.g., four, motors which are located in pairs in wings (to be described) of the insole 22. The insole is arranged to be placed within any item of footwear, e.g., a shoe, boot, sandal, etc., (not shown) so that it underlies the sole of the person wearing the footwear. The insole includes two wings that extend outward from the medial and lateral sides of the insole adjacent the arch region. The asynchronous vibration inducing mechanisms, e.g., electric motors, are disposed at the wings, e.g., installed in the insole at the location of the wings. When the insole is in place in the footwear these wings curve upward around the lateral and medial sides of the person's foot so that his/her weight is not is not resting on the wing portions, nor on the motors contained therein.

Moreover, the motors of the vibration inducing mechanism are located in the wings so that they are closely adjacent the Sural Nerve and the Medial Plantar Nerve, respectively. It is believed that providing the source of vibration at these locations has the beneficial effect of increased pain reduction.

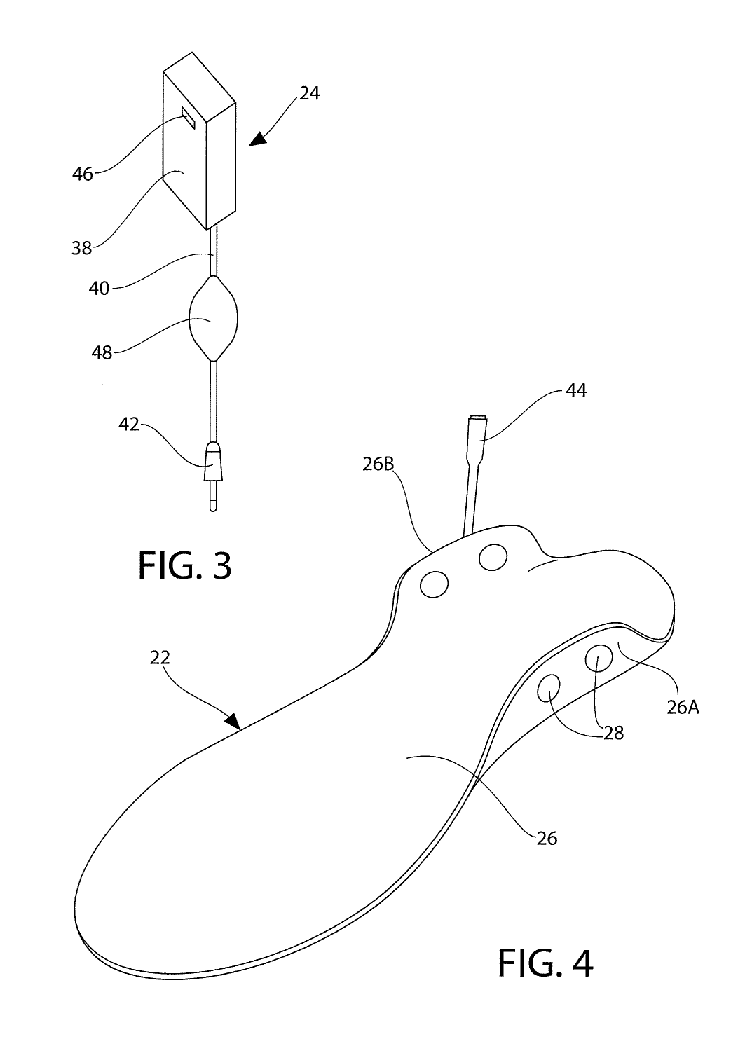

The insole is constructed of a material that is thin, conformable and compliant, such that it can easily fit into a shoe or other item of footwear in the interest of wearer comfort. The insole is constructed to exhibit the ability to readily transmit or propagate the vibration produced by the motors to the remainder of the insole to apply that vibration across virtually the entire expanse of the person's foot, i.e., heel to toe, side-to-side. Power for the motors is provided from the power source 24 (FIG. 3), which will be described later and which is preferably located remote from the insole, but is connected to it via electrical cables.

In order to get the benefits of the invention all that a person has to do is to insert the insoles into his/her footwear and then put the footwear on his/her feet. The vibration inducing mechanisms can be turned on when desired, e.g., while the person is at rest, such as sitting or resting, or while the person is standing or otherwise ambulatory. The vibration produced when the vibratory mechanisms are operated is significant and easily felt by the person across virtually the entire foot.

As best seen in FIG. 1 the vibrating insole 20 basically comprises a thin, member whose profile is such that it encompasses virtually the entire sole of a person's foot (although it could be less extensive, e.g., be limited to a particular area or region of the wearer's sole). To that end, it consists of a central portion 26 and a pair of wing portions 26A and 26B extending from the lateral and medial sides, respectively, of the central portion 26. Two pairs of vibration inducing motors 28 are mounted in the wing portions 26A and 26B. When installed in the footwear the wing portions 26A and 26B curve upward, like shown in FIG. 4, so that they are positioned on the lateral and medial sides of the person's foot adjacent the arch region. As such the person does not put his/her weight on the motors. It should be pointed out at this juncture that each wing may include only a single motor or more than two motors, if desired. In fact, both wings do not need to contain any motor, as long as the other wing contains a motor.

The details of the insole 22 will now be described with reference to FIG. 2. As can be seen therein the insole 20 is composed of plural layers 32, 34 and 36. The layer 32 is the principal layer and is located between the layers 34 and 36. The layer 32 is made from a material that is conformable so that it can fit easily into the footwear. Layer 32 should have good vibration transmission/propagation properties since the vibration motors 28 are contained in the wings 26A and 26B and it is desirable to transmit easily felt vibrations all the way from the vibration motors to the bottom of the toes, forefoot and heel. One type of material that has been found particularly suitable for layer 32 is a resin bonded nonwoven layer. An example of a resin bonded nonwoven with suitable properties is Bear-Tex Ultra fine abrasive sheets, made by Norton Company and available from Manhattan Supply Company, PN 70501119. Those sheets consist of a non-woven web of nylon fibers that are bonded together with a synthetic resin. The sheets contain a significant percentage of open space between fibers filled only with air. The sheets are conformable and resilient. The outer layers 34 and 36 of the insole constitute thin covering layers of polyester suede that may be adhesively coupled to the inner layer 32 and are of a texture suitable for use in an insole.

The vibration inducing mechanisms 28 are any suitable transducer capable of producing vibratory energy or motion when actuated. In the exemplary embodiment the mechanism comprise plural motors, e.g., Sunon DC brushless vibration motors VRB 1434. These motors are coin style vibration motors and typically run at about 8000 RPM off of a DC source of power of approximately 3 volts. Other vibration inducing mechanisms/motors may be used. The motors preferably provide vibrations from 1 Hz to 500 Hz, but other frequencies can be used as well.

In FIG. 3 the power source for powering the motors 28 is shown. To that end, the power source basically comprises a housing or box 38 containing 3 AAA rechargeable batteries (not shown) that are 1.2 volts each. Current from the batteries is delivered from the batteries to the motors via a cable 40 and an associated electrical connector 42. The connector 42 is arranged to be releasably connected to another electrical connector 44 (FIGS. 1 and 4) which forms the electrical input to the motors 28 of the insole 22. The housing 38 includes an ON/OFF switch 46 to connect/disconnect the power from the batteries to the motors. Preferably the housing includes means for mounting it to the leg just below the knee or just above the ankle of the person. To that end, the housing includes an adhesively mounted patch of a multi-hook fastener material, such as Velcro® (not shown), on it. A cooperating loop strap 50 of Velcro® can be wrapped around the person's leg just below the knee or above the ankle (like shown in FIG. 6) and the housing 38 can be releasably coupled to that strap by the Velcro® patch on the housing. The device 20 also may include a retractor 48, which serves to roll up any excess cable, so that the cable length is adjustable.

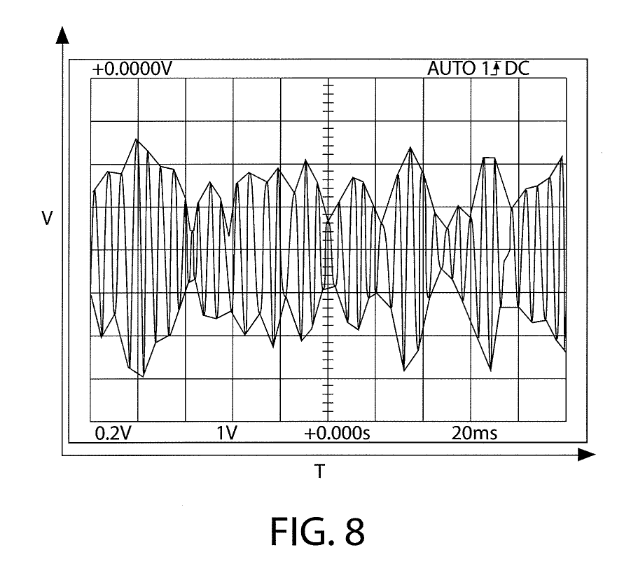

It should be pointed out at this juncture, that while the motors 28 are connected in parallel to the power source, due to their construction the vibrations produced by each individual motor in response to a particular voltage applied thereto varies from motor to motor. Thus, the application of any particular voltage to all of the motors will have the effect of each of the motors vibrating at a somewhat different frequency. Thus, the cumulative effect of the combination of those multiple vibration components will be a vibrations simultaneously occurring at a broad range of frequencies so that the resulting vibrations fluctuate in amplitude. This action is believed to have the beneficial effect of stimulating the various mechanoreceptors and pathways that are responsive to different frequencies. Moreover, the motors tend to drift somewhat in their output, so that the vibration produced by them changes over time. All of those effects tend to randomize the vibrations, i.e., produce asynchronous vibrations, which are propagated across the sole of the user's foot. In testing an exemplary embodiment of an insole constructed in accordance with this invention the asynchronous vibration exhibited frequency components or beats of 250 Hz, 20 Hz and 6 Hz and amplitudes having peaks of 0.6 volts, 3.7 volts and 2.2 volts, when the motors were driven with a nominal voltage of 3 volts. In FIG. 8 there is shown a portion of any exemplary waveform representing the resulting asynchronous vibration produced by an exemplary device constructed in accordance with this invention. In that graph the abscissa represents time, while the ordinate represents voltage (which is proportional to the measured acceleration of the insole). As can be seen the envelope of the vibration (shown by the white lines) is random and varies in amplitude. This asynchronous or randomized vibration is believed to maximize the device's pain relieving ability by preventing the likelihood of a user adapting to the vibration. In addition, the use of the device in a closed shoe or other footwear increases the magnitude of vibration to the foot through the principle of coupling. Finally, because device is designed to be used while a person is mobile, it maximizes the pain relief potential by combining vibration and movement.

It must be point out at this juncture that other means for producing asynchronous vibrations and for propagating them across the foot of the wearer are contemplated by this invention. Thus, one could program the motors to run in a random pattern or in a “pseudo-random” pattern, which while not precisely random, still feels random by the user because the pattern is very long. Moreover, other devices than electrical motors, can be used to create the asynchronous vibrations.

To use the device, all that is required of a person is to place the insoles in the person's footwear. The power housing 38 can be secured about the leg just below the knee or about the wearer's ankle as shown in FIG. 6. The connector 42 can then be connected to connector 44. When it is desired to start the vibratory action, all that is required is to switch the power on by the ON/OFF switch, whereupon the motors 28 begin to vibrate. The vibration is transferred or propagated from those motors through the wings to the remainder of the insole via the layer 32, whereupon the vibration is produced across the entire expanse of the person's foot.

The invention may be used while sitting, standing or walking. The insoles are configured such that they do not have to be removed when there is no vibration treatment. The user simply turns them off. The user can then easily and conveniently turn them back on at a time when additional pain relief is required. The insoles can remain in the footwear when the footwear is removed so that they are readily available when the footwear is put on again.

The forgoing vibration treatment has been found to be especially helpful for relieving peripheral neuropathic pain in the feet and lower legs. In this regard, it is believed that vibration provides a signal to the nerves that blocks the chronic pain signal that can be associated with peripheral neuropathy thereby providing pain relief to the user. Individuals have reported a reduction in pain of 90% immediately after starting the vibration treatment. The pain relief may often be sustained for a period of hours after the vibration treatment has stopped, although the reasons for this sustained effect are not known.

In summary, the devices constructed in accordance with this invention combine essential features of vibrational stimulation into one system. Moreover, they are designed with the user in mind. In this regard, when the device is in the form of an insole, the soft conforming insole material offers a comfortable surface for the user, while transmitting vibration through the entire length of the insert. Additionally, the vibration transducers are not mounted under the user's feet; rather, the transducers are positioned medial and lateral to the foot in order to reduce pressure points, but the material making up the insole is such as to effectively propagate the vibrations across the entire sole of the wearer's foot. In this regard, the chart of FIG. 7 shows the transmittance of vibration through an insole constructed in accordance with this invention as compared to an insole constructed of standard gel and an insole constructed of standard air-foam.

The subject invention is also believed to provide users, particularly those with peripheral neuropathy, with an improved ability to sense with their feet and thus enhance their balance. As a result they appear to be less susceptible to falling. Moreover, these beneficial effects appear to be residual. In particular, some users of devices constructed in accordance with this invention report improvements in balance during times when the device is actually vibrating as well as times after they have had it on for a while and when they have been using it daily over the course of several days to weeks.

Without further elaboration the foregoing will so fully illustrate our invention that others may, by applying current or future knowledge, adopt the same for use under various conditions of service.

We claim:

1. A device for applying vibration to the foot of a person comprising a member arranged for disposition within an item of footwear to be worn by a person to apply vibration to the foot of the person wearing the footwear, said device comprising a thin base member and an asynchronous vibration inducing mechanism, said thin body member being formed of a material that exhibits good vibration transmission characteristics and having a medial side, a lateral side and a first wing portion extending from one of said medial side or said lateral side, said base member being arranged to be disposed under the sole of the person's foot with said first wing portion being located beside a respective portion of the person's foot but not under the sole of the wearer's foot, said asynchronous vibratory inducing mechanism being coupled to said first wing portion to cause said first wing portion to vibrate asynchronously, whereupon said asynchronous vibration of said first wing portion is transmitted to said thin base member and propagated thereby to apply asynchronous vibration across the sole of the wearer's foot.

2. The device of claim 1 wherein said thin body member is conformable and compliant to readily fit into the item of footwear.

3. The device of claim 1 wherein said asynchronous vibration inducing mechanism comprises plural motors arranged to be driven to cause them to vibrate.

4. The device of claim 3 wherein said base member includes a second wing portion extending from the other of said medial side or said lateral side, and wherein at least one of said motors is located in said first wing member and at least another of said motors is located in said second wing member, whereupon the vibration produced by said at least one of said motors and said at least another of said motors is transmitted from the associated wing portion to said base member at which of said second wing portion is transmitted to said thin body member and propagated thereby to apply asynchronous vibration to the sole of the wearer's foot.

5. The device of claim 1 wherein said thin body member is formed of a resin bonded non-woven layer.

6. The device of claim 4 wherein said non-woven layer comprises a web of nylon fibers that are bonded together with a synthetic resin.

7. The device of claim 3 wherein each of said motors comprises an electrical vibration motor.

8. The device of claim 7 wherein each of said electrical vibration motors is brushless.

9. The device of claim 8 wherein said each of said electrical vibration motors is a coin style motor.

10. The device of claim 7 additionally comprising an electrical power source located externally of the item of footwear for causing said electrical vibration motors to operate.

11. The device of claim 3 wherein said device additionally comprises an electrical power source for applying at least one voltage to said plural motors, whereupon said motors asynchronously produce vibrations of varying frequencies and amplitudes.

12. The device of claim 11 wherein the voltage applied to said motors from said power and control source is adjustable.

13. The device of claim 1 wherein each of said motors is vibrated at a frequency in the range of 1 Hz to 500 Hz, with at least some of said motors operating at different frequencies within said range.

14. The device of claim 12 wherein each of said motors is vibrated at a frequency in the range of 1 Hz to 500 Hz, with at least some of said motors operating at different frequencies within said range.

15. A method of providing asynchronous vibration to the foot of a person wearing an item of footwear comprising:
(A) providing an device comprising a thin base member and an asynchronous vibration inducing mechanism, said thin base member being formed of a material that exhibits good vibration transmission characteristics and having a medial side, a lateral side and a first wing portion extending from one of said medial side or said lateral side;
(B) disposing said base member under the sole of the person's foot, with said first wing portion being located beside a respective portion of the wearer's foot, but not under the sole of the person's foot, said asynchronous vibration inducing mechanism being coupled to said first wing portion; and
(C) operating said asynchronous vibration inducing mechanism to cause said first wing portion to vibrate, whereupon said vibration of said first wing portion is transmitted to said thin base member and propagated thereby to apply asynchronous vibration across the sole of the wearer's foot.

16. The method of claim 15 wherein asynchronous vibration inducing mechanism comprises plural motors and wherein said base member includes a second wing portion extending from the other of said medial side or said lateral side, with at least one of said motors being located in said first wing member and at least another of said motors being located in said second wing member, and wherein said method additionally comprises:
(D) operating said at least one and said at least another of said motors to cause said first and second wing portions to vibrate, whereupon the vibrations of said wing portions are transmitted to said thin base member and propagated thereby to apply vibration across the sole of the wearer's foot.

17. The method of claim 15 wherein each of said motors is vibrated at a frequency in the range of 1 Hz to 500 Hz, with at least some of said motors operating at different frequencies within said range.

18. The method of claim 15 wherein said method is used for treating peripheral neuropathic pain.

19. The method of claim 16 additionally comprising the step of the person wearing the item of footwear while being ambulatory.

20. A method for improving the sense of feel and balance of a person having peripheral neuropathy comprising providing vibration to the person's foot at a level which is perceptible by the person.

21. The method of claim 20 wherein the application of vibration to the person's foot is accomplished by:
(A) providing a device comprising a thin base member and an asynchronous vibration inducing mechanism;
(B) disposing said base member under the sole of the person's foot;
(C) operating said asynchronous vibration inducing mechanism to transmit asynchronous vibration to said base member and to propagate the asynchronous vibration across the base member and thereby apply asynchronous vibration across the sole of the wearer's foot.

22. The method of claim 21 wherein of said asynchronous vibration inducing mechanism includes plural motors which operate at a frequency in the range of 1 Hz to 500 Hz, with at least some of said motors operating at different frequencies within said range.

23. The method of claim 21 wherein the application of said asynchronous vibration causes the sense of feel and balance to persist beyond the time that the asynchronous vibration is applied.
